(19) United States
(12) Patent Application Publication
(10) Pub. No.: US 2016/0327543 A1
(43) Pub. Date: Nov. 10, 2016

(54) METHOD FOR CONTINUOUSLY MONITORING CHEMICAL OR BIOLOGICAL PROCESSES

(71) Applicant: John P. JASPER, Niantic, CT (US)

(72) Inventor: John P. JASPER, Niantic, CT (US)

(21) Appl. No.: 15/108,596

(22) PCT Filed: Dec. 30, 2014
(86) PCT No.: PCT/US2014/072644

§ 371 Date: Jun. 28, 2016

Related U.S. Application Data
(60) Provisional application No. 61/923,175, filed on Jan. 2, 2014.
(60) Provisional application No. 62/010,755, filed on Jun. 11, 2014.
(60) Provisional application No. 62/082,499, filed on Nov. 20, 2014.

(51) Int. Cl. G01N 33/50; H01J 49/26; G01N 24/08; G01N 33/15; G01N 21/39

(52) U.S. Cl. CPC G01N 33/50; G01N 33/15; G01N 21/39; G01N 24/088; H01J 49/26; G01N 2021/391

(57) ABSTRACT

Methods and systems are provided for continuously monitoring the progress of chemical or biological processes. These methods and systems utilize isotopic information for one or more isotope ratios from elements present in samples from these processes. Also provided are methods for continuously assessing the yield of chemical or biological processes and methods for continuously monitoring the fraction of reactants remaining in chemical or biological processes.

### RELATED APPLICATIONS

This application claims priority to U.S. Provisional Patent Application No. 61/923,175 filed on Jan. 2, 2014, U.S. Provisional Patent Application No. 62/010,755 filed on Jun. 11, 2014, and U.S. Provisional Patent Application No. 62/082,499 filed on Nov. 20, 2014, the disclosures of each of which are incorporated by reference herein in their entirety.

### FIELD OF THE INVENTION

The present invention relates to methods for continuously monitoring the progress of chemical or biological processes, such as chemical or biological reactions. More particularly, the present invention relates to methods for continuously monitoring the progress of chemical or biological processes utilizing isotopic information via one or more isotope ratios from elements present in samples involved in these processes. The present invention also relates to systems for continuously monitoring the progress of these processes.

### BACKGROUND OF THE INVENTION

A wide range of chemical and biological processes are used to manufacture chemicals, pharmaceuticals, biologics, polymers, food stuffs, fuels, and most other products in the modern world. Some of these processes are conducted in discreet batches or steps, whereas other processes are conducted on a continuous basis, often using feedstock streams and producing output streams containing the final products. In the case of a continuous process, it would be highly desirable to continuously monitor the progress or yield of that process, or to monitor the fraction of reactants remaining. In the case of a batch process it would also be highly desirable to continuously monitor the progress or yield of that process, or to monitor the fraction of reactants remaining. The present invention provides such methods for continuously monitoring the progress or yield of chemical or biological processes or for monitoring the fraction of reactants remaining. These methods utilize isotopic information for one or more isotope ratios from elements present in samples sampled from these processes.

Large-scale scientific research involving both radioactive and non-radioactive (i.e. stable) isotopes goes back to the time of the Manhattan Project, which produced the first atomic bombs during World War II. Isotopes—whether stable or radioactive—are forms of the same chemical element having different atomic masses. For example, uranium has an isotopic form with a mass of 235 (uranium-235 or ²³⁵U) and also an isotopic form with a mass of 238 (uranium-238 or ²³⁸U). Although radioactive isotopes can be used in the methods and systems of the present invention, the methods and systems herein are focused primarily on non-radioactive, stable isotopes.

By 1942, isotopes had only been known for about thirty years. Most of the research involving isotopes had been theoretical, relating to determining atomic structures and studying the then-mysterious properties of radioactivity. The Manhattan Project changed all that. The dire urgency of the war effort led to the development of sophisticated techniques for separating and identifying isotopes. One of these techniques, Isotope Ratio Mass Spectrometry (or IRMS for short), which is used to measure the relative abundance of isotopes in a sample, is now an important tool for studying and using isotopes.

Furthermore, the stable isotopic composition of matter has been recognized since about 1945 as a criterion for highly-specifically differentiating one material from another with the same elemental composition. In the field of geochemical oil exploration and prospecting, measurement of the isotopic compositions of large numbers of individual organic compounds of oil samples from various oil reservoirs have assisted in clarifying the origin of specific compounds correlating the organic compounds with particular petroleum sources, recognizing the existence of multiple petroleum sources, examining the mechanisms of petroleum generation, source mixing, and improving the sensitivity of petroleum migration studies. This information, particularly in connection with seismological data, can be used to predict locations of other oil reservoirs to which oil may have migrated from a common source of generation or formation.

Isotope ratio monitoring has had further applications in the biomedical field, wherein non-radioactive and stable isotopes are used as tracer labels in drug metabolism and other biomedical studies where natural variations in isotopic abundances may also carry additional information regarding sources and fates of metabolites. Additionally, radioactive and stable isotopic labeling apparatus and methods in the medical fields employ typically costly labeled compounds having isotope ratios much different than those found in natural abundance.

Methods and systems for isotope identification are described in U.S. Pat. No. 7,323,341 B1, to Jasper, issued Jan. 29, 2008 and U.S. Pat. No. 8,367,414 B2, to Jasper, issued Feb. 5, 2013, which are incorporated by reference herein in their entirety. U.S. Pat. No. 7,323,341 states that it relates to a stable isotopic identification and method for identifying products using naturally occurring isotopic concentrations or isotopic ratios in products, especially in the pharmaceutical industry, and more particularly to an identification and a method utilizing such isotopic concentrations or ratios in a machine readable form for identifying products and tracking products through manufacturing, marketing and use of a product, and readily indexing product information to the product. U.S. Pat. No. 8,367,414 states that it relates to the field of isotope analysis and, in particular, an emerging new field of analytical chemistry that is directed to the derivation of information regarding the origins of synthetic products from processes in which the amounts or ratios of isotopes in either synthetic starting materials, intermediates or products are traced.

Methods for characterizing manufacturing pathways using isotopic methods have also been described. See, e.g., J. P. Jasper, L. E. Weaner, and J. M. Hayes, Process Patent Protection: Characterizing Synthetic Pathways by Stable-Isotope Measurements, Pharmaceutical Technology, 2007, 31(3):68-73, which is incorporated by reference herein in its entirety. This reference describes methods by which precise analyses of stable-isotopic abundances can be used in security and forensic applications for pharmaceutical materials. These methods include product and process authentication of raw materials, pharmaceutical intermediates, drug substances, formulated drug products, and synthetic pathways. Since the inception of these techniques, there have been yet further improvements in isotope ratio monitoring sensitivity and precision, as well as frequency, and a reduction in sample size requirements.

In the combustible fuel, environmental, food, explosives, ammunition, polymer, paint, and coating industries, continuous isotope identification methods would be useful for continuously and cost-effectively monitoring manufacturing processes. In the pharmaceutical industry, there is a need to trace ingredients through the manufacturing process, through the marketplace, and into various usages. In many instances, it would be highly desirable to have continuous isotope monitoring methods, to monitor chemical and biological processes for making active pharmaceutical ingredients (APIs) and finished pharmaceutical products, and for making biological products.

It is apparent from the above there is an ongoing need for new methods and systems for continuously monitoring a wide array of chemical and biological processes.

### BRIEF DESCRIPTION OF THE FIGURES

FIG. 1 depicts the isotopic composition of a reaction product plotted as a function of reaction yield. The isotopic composition, δ, increases as the reaction yield approaches 1, i.e. as the reaction approaches completion. The symbol, ‰, designates permil or what is also referred to as parts per thousand. The reactant or reactants, the process, and the product or products are indicated on the plot.

FIG. 2 depicts the carbon-isotopic composition (δ¹³C) of synthetic intermediates (C, E, G) and the final product, I, as a function of reaction step with (upper line) and without (lower line) the contributions of partial-reaction completion (f) and isotopic fractionation (ε).

FIG. 3 depicts the carbon-isotopic differences between isotopic compositions predicted and those which would be observed in the absence of isotope effects, δ_{P}*−δ_{P}, ‰, corresponding to the values in Table 1 as presented in the patent application.

FIG. 4 depicts a system for continuously monitoring the progress of a chemical or a biological process, in which a gaseous product (or by-product, e.g., CO₂) is generated. This system illustrates a stirred reactor vessel and a sampling device, which in this case is a carrier gas line for blowing a carrier gas through or over the chemical or biological process to continuously sample the chemical or biological process or to collect the gaseous product or by-product. The system also depicts an effluent tube, which is a part of the sampling device, which feeds in to an isotope analyzer and an associated computerized data system (CDS). The interface is essentially the connection between the sampling device (in this case the effluent tube) and the isotope analyzer.

FIG. 5 depicts graphically model equations for CO₂ flow, Δδ, and integrated yield for a beer fermentation process in which CO₂ is a by-product of the fermentation process.

### SUMMARY OF THE INVENTION

The present invention relates to methods for continuously monitoring the progress of chemical or biological processes, such as chemical or biological reactions. More particularly, the present invention relates to methods for continuously monitoring the progress of chemical or biological processes utilizing isotopic information for one or more isotope ratios from elements present in samples from these processes. The present invention also relates to systems for continuously monitoring the progress of these processes.

### DETAILED DESCRIPTION OF THE INVENTION

The present invention relates to methods for continuously monitoring the progress of chemical or biological processes.

In one aspect the present invention relates to a method for continuously monitoring the progress of a chemical process or a biological process comprising the steps of:
(a) sampling the process at a selected frequency to obtain one or more samples from the process,
(b) determining or assessing the isotopic information for one or more isotope ratios from elements present in each sample,
(c) processing the information to determine the isotopic information as a function of time, and
(d) determining or assessing the progress of the process from the information processed in step (c).

In another aspect the present invention relates to a method for continuously monitoring the progress of a chemical or biological process comprising the steps of:
(a) initially sampling the process at a first time point (i.e. an initial time point),
(b) determining or assessing the isotopic information for one or more isotope ratios from elements present in the sample obtained at the first time point (i.e. the initial time point),
(c) further sampling the process at one or more time points after the first time point (i.e. the initial time point),
(d) determining or assessing the isotopic information for one or more isotope ratios from elements present in each sample obtained after the first time point (i.e. the initial time point), and
(e) comparing the isotopic information from each sample obtained after the first time point (i.e. the initial time point) with the isotopic information from the sample obtained at the first time point (i.e. the initial time point) to determine or assess the progress of the process.

In another aspect the present invention relates to a method for continuously assessing the yield of a chemical process or a biological process comprising the steps of:
(a) sampling the process at a selected frequency to obtain one or more samples from the process,
(b) determining or assessing the isotopic information for one or more isotope ratios from elements present in each sample,
(c) processing the information to determine the isotopic information as a function of time, and
(d) determining or assessing the yield of the process from the information processed in step (c).

In another aspect the present invention relates to a method for continuously assessing the yield of a chemical process or a biological process comprising the steps of:
(a) initially sampling the process at a first time point (i.e. an initial time point),
(b) determining or assessing the isotopic information for one or more isotope ratios from elements present in the sample obtained at the first time point (i.e. the initial time point),
(c) further sampling the process at one or more time points after the first time point (i.e. the initial time point),
(d) determining or assessing the isotopic information for one or more isotope ratios from elements present in each sample obtained after the first time point (i.e. the initial time point), and
(e) comparing the isotopic information from each sample obtained after the first time point (i.e. the initial time point) with the isotopic information from the sample obtained at the first time point (i.e. the initial time point) to determine or assess the yield of the process.

In another aspect the present invention relates to a method wherein the yield is an incremental yield.

In another aspect the present invention relates to a method wherein the yield is an instantaneous yield.

In another aspect the present invention relates to a method for continuously monitoring the progress to the end point of a chemical process or a biological process comprising the steps of:
(a) sampling the process at a selected frequency to obtain one or more samples from the process,
(b) determining or assessing the isotopic information for one or more isotope ratios from elements present in each sample,
(c) processing the information to determine the isotopic information as a function of time, and
(d) determining or assessing the progress of the process to the end-point of the process from the information processed in step (c).

In another aspect the present invention relates to a method for continuously monitoring the progress to the end point of a chemical process or a biological process comprising the steps of:
(a) initially sampling the process at a first time point (i.e. an initial time point),
(b) determining or assessing the isotopic information for one or more isotope ratios from elements present in the sample obtained at the first time point (i.e. the initial time point),
(c) further sampling the process at one or more time points after the first time point (i.e. the initial time point),
(d) determining or assessing the isotopic information for one or more isotope ratios from elements present in each sample obtained after the first time point (i.e. the initial time point), and
(e) comparing the isotopic information from each sample obtained after the first time point (i.e. the initial time point) with the isotopic information from the sample obtained at the first time point (i.e. the initial time point) to determine or assess the progress to the end-point of the process.

In another aspect the present invention relates to a method for continuously monitoring the fraction of a reactant remaining in a chemical process or a biological process comprising the steps of:
(a) sampling the process at a selected frequency to obtain one or more samples from the process,
(b) determining or assessing the isotopic information for one or more isotope ratios from elements present in each sample,
(c) processing the information to determine the isotopic information as a function of time, and
(d) determining or assessing the amount of the reactant remaining from the information processed in step (c).

In another aspect the present invention relates to a method for continuously monitoring the fraction of a reactant remaining in a chemical process or a biological process comprising the steps of:
(a) initially sampling the process at a first time point (i.e. an initial time point),
(b) determining or assessing the isotopic information for one or more isotope ratios from elements present in the sample obtained at the first time point (i.e. the initial time point),
(c) further sampling the process at one or more time points after the first time point (i.e. the initial time point),
(d) determining or assessing the isotopic information for one or more isotope ratios from elements present in each sample obtained after the first time point (i.e. the initial time point), and
(e) comparing the isotopic information from each sample obtained after the first time point (i.e. the initial time point) with the isotopic information from the sample obtained at the first time point (i.e. the initial time point) to determine or assess the amount of the reactant remaining.

In another aspect the present invention relates to a method for continuously monitoring the progress of a chemical process or a biological process comprising the steps of:
(a) continuously sampling the process,
(b) continuously determining or assessing the isotopic information for one or more isotope ratios from elements present in each sample or the continuous samplings,
(c) continuously processing the information to determine the isotopic information as a function of time, and
(d) continuously determining or assessing the progress of the process from the information processed in step (c).

In another aspect the present invention relates to a method for continuously assessing the yield of a chemical process or a biological process comprising the steps of:
(a) continuously sampling the process,
(b) continuously determining or assessing the isotopic information for one or more isotope ratios from elements present in each sample or from the continuous samplings,
(c) continuously processing the information to determine the isotopic information as a function of time, and
(d) continuously determining or assessing the yield of the process from the information processed in step (c).

In another aspect the present invention relates to a method for continuously monitoring the progress to the end point of a chemical process or a biological process comprising the steps of:
(a) continuously sampling the process,
(b) continuously determining or assessing the isotopic information for one or more isotope ratios from elements present in each sample or from the continuous sampling,
(c) continuously processing the information to determine the isotopic information as a function of time, and
(d) continuously determining or assessing the progress of the process to the end-point of the process from the information processed in step (c).

In another aspect the present invention relates to a method for continuously monitoring the fraction of a reactant remaining in a chemical process or a biological process comprising the steps of:
(a) continuously sampling the process,
(b) continuously determining or assessing the isotopic information for one or more isotope ratios from elements present in each sample or from the continuous sampling,
(c) continuously processing the information to determine the isotopic information as a function of time, and
(d) continuously determining or assessing the amount of the reactant remaining from the information processed in step (c).

In another aspect the present invention relates to a method for continuously monitoring the progress of a chemical process or a biological process; or to a method for continuously assessing the yield of a chemical process or a biological process; or to a method for continuously monitoring the progress to the end point of a chemical process or a biological process; or to a method for continuously monitoring the fraction of a reactant remaining in a chemical process or a biological process; wherein the chemical process or the biological process consumes a gaseous reactant or produces a gaseous product or byproduct, which in other aspects of the present invention gaseous reactant, product, or byproduct is selected from CO₂, CO, and mixtures thereof, comprising the steps of:
(a) continuously sampling the gaseous product or byproduct from the process,
(b) continuously determining or assessing the isotopic information for one or more isotope ratios from elements present in each sample or from the continuous sampling,
(c) continuously processing the information to determine the isotopic information as a function of time, and as applicable, and
(d) continuously determining or assessing the progress of the process from the information processed in step (c); or determining or assessing the yield of the process from the information processed in step (c); or determining or assessing the amount of the reactant remaining from the information processed in step (c); or determining or assessing the progress of the process to the end-point of the process from the information processed in step (c); or determining or assessing the amount of the reactant remaining from the information processed in step (c).

In another aspect the present invention relates to a method for continuously monitoring the progress of a chemical process or a biological process which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

In another aspect, the present invention relates to a method for continuously assessing the yield of a chemical process or a biological process which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

In another aspect the present invention relates to a method for continuously monitoring the progress to the end point of a chemical process or a biological process which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

In another aspect the present invention relates to a method for continuously monitoring the fraction of a reactant remaining in a chemical process or a biological process, which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

In another aspect the present invention relates to a method for continuously monitoring the proportions of two or more products produced in a chemical process or a biological process comprising the steps of:
(a) continuously sampling the products from the process,
(b) continuously determining or assessing the isotopic information for one or more isotope ratios from elements present in each sample from the products or from the continuous sampling of the products,
(c) continuously processing the information to determine the isotopic information as a function of time, and
(d) continuously determining or assessing the proportions of two or more products from the information processed in step (c).

In another aspect the present invention relates to a method wherein the proportion is an incremental proportion.

In another aspect the present invention relates to a method wherein the proportion is an instantaneous proportion.

In another aspect the present invention relates to a method wherein the chemical process or the biological process is a chemical process.

In another aspect the present invention relates to a method wherein the chemical process is a chemical reaction.

In another aspect the present invention relates to a method wherein the chemical reaction is a batch chemical reaction.

In another aspect the present invention relates to a method wherein the chemical reaction is a continuous chemical reaction.

In another aspect the present invention relates to a method according wherein the continuous chemical reaction is a flow chemical reaction.

In another aspect the present invention relates to a method wherein the chemical process or the chemical reaction is utilized for the manufacture of a pharmaceutical product.

In another aspect the present invention relates to a method wherein the chemical process or the biological process is a biological process.

In another aspect the present invention relates to a method wherein the biological process is a biological reaction.

In another aspect the present invention relates to a method according wherein the biological reaction is a batch biological reaction.

In another aspect the present invention relates to a method wherein the biological reaction is a continuous biological reaction.

In another aspect the present invention relates to a method wherein the continuous biological reaction is a flow biological reaction.

In another aspect the present invention relates to a method wherein the biological process or the biological reaction is utilized for the manufacture of a biological product.

In another aspect the present invention relates to a method wherein the process is a biological process such as a fermentation process.

In another aspect the present invention relates to a method wherein the fermentation process is a beer fermentation process.

In another aspect the present invention relates to a method wherein the elements are selected from elements that have two or more isotopes.

In another aspect the present invention relates to a method wherein the elements are selected from elements that have two or more stable isotopes.

In another aspect the present invention relates to a method wherein the elements are selected from elements that have two or more naturally-occurring stable isotopes.

In another aspect the present invention relates to a method wherein the elements are selected from hydrogen, carbon, nitrogen, oxygen, sulfur, chlorine, bromine, and combinations thereof.

In another aspect the present invention relates to a method wherein the isotopes are stable isotopes.

In another aspect the present invention relates to a method wherein the isotopes are naturally-occurring stable isotopes.

In another aspect the present invention relates to a method where the isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In another aspect the present invention relates to a method wherein the isotope ratios are selected from the following pairs of isotopes: ¹H and ²H, ¹²C and ¹³C, ¹⁴N and ¹⁵N, ¹⁶O, and ¹⁸O, ³²S and ³⁴S, ³⁵Cl and ³⁷Cl, and ⁷⁹Br, and ⁸¹Br, and combinations thereof.

In another aspect the present invention relates to a method wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br, and combinations thereof.

In another aspect the present invention relates to a method wherein the isotope ratio is ²H/¹H.

In another aspect the present invention relates to a method wherein the isotope ratio is ¹³C/¹²C.

In another aspect the present invention relates to a method wherein the isotope ratio is ¹⁵N/¹⁴N.

In another aspect the present invention relates to a method wherein the isotope ratio is ¹⁸O/¹⁶O.

In another aspect the present invention relates to a method wherein the isotope ratio is ³⁴S/³²S.

In another aspect the present invention relates to a method wherein the isotope ratio is ³⁷Cl/³⁵Cl.

In another aspect the present invention relates to a method wherein the isotope ratio is ⁸¹Br/⁷⁹Br.

In another aspect the present invention relates to a method wherein the isotopic information is intrinsic isotopic information.

In another aspect the present invention relates to a method wherein the isotopic information is obtained from CO₂ or CO produced from the process.

In another aspect the present invention relates to a method wherein the isotopic information is obtained from CO₂ produced by the process

In another aspect the present invention relates to a method wherein the isotopic information is obtained from CO produced by the process.

In another aspect the present invention relates to a method wherein the isotopic information is obtained from the isotope ratio of the ¹³C/¹²C or the ratio of ¹⁸O/¹⁶O, or combinations of these ratios from CO₂ or CO produced from the process.

In another aspect the present invention relates to a method wherein the isotopic information is obtained from the isotope ratio of the ¹³C/¹²C from the CO₂ or CO produced from the process.

In another aspect the present invention relates to a method wherein the isotopic information is obtained from the isotope ratio of the ¹³C/¹²C from the CO₂ produced from the process.

In another aspect the present invention relates to a method wherein the isotopic information is obtained from the isotope ratio of the ¹³C/¹²C from the CO produced from the process.

In another aspect the present invention relates to a method wherein the isotopic information is obtained from the isotope ratio of the ¹⁸O/¹⁶O from the CO₂ or CO produced from the process.

In another aspect the present invention relates to a method wherein the isotopic information is obtained from the isotope ratio of the ¹⁸O/¹⁶O from the CO₂ produced from the process.

In another aspect the present invention relates to a method wherein the isotopic information is obtained from the isotope ratio of the ¹⁸O/¹⁶O from the CO produced from the process.

In another aspect the present invention relates to a system for continuously monitoring the progress of a chemical process or a biological process comprising:

(a) a device for sampling the process,

(b) an interface,

(c) an isotope analyzer, and

(d) a computerized data system (CDS).

In another aspect the present invention relates to a system for continuously monitoring the progress of a chemical process or a biological process comprising:

(a) a reactor,

(b) a device for sampling the process,

(c) an interface,

(d) an isotope analyzer, and

(e) a computerized data system (CDS).

In another aspect the present invention relates to a system for determing or assessing the yield of a chemical process or a biological process comprising:

(a) a device for sampling the process,

(b) an interface,

(c) an isotope analyzer, and

(d) a computerized data system (CDS).

In another aspect the present invention relates to a system for determining or assessing the yield of a chemical process or a biological process comprising:

(a) a reactor,

(b) a device for sampling the process,

(c) an interface,

(d) an isotope analyzer, and

(e) a computerized data system (CDS).

In another aspect the present invention relates to a system wherein the yield is an incremental yield.

In another aspect the present invention relates to a system wherein the yield if an instantaneous yield.

In another aspect the present invention relates to a system for continuously monitoring the progress to the end-point of a chemical process or a biological process comprising:

(a) a device for sampling the process,

(b) an interface,

(c) an isotope analyzer, and

(d) a computerized data system (CDS).

In another aspect the present invention relates to a system for continuously monitoring the progress to the end-point of a chemical process or a biological process comprising:

(a) a reactor,

(b) a device for sampling the process,

(c) an interface,

(d) an isotope analyzer, and

(e) a computerized data system (CDS).

In another aspect the present invention relates to a system for continuously monitoring the fraction of a reactant remaining in a chemical process or a biological process comprising:

(a) a device for sampling the process,

(b) an interface,

(c) an isotope analyzer, and

(d) a computerized data system (CDS).

In another aspect the present invention relates to a system for continuously monitoring the fraction of a reactant remaining in a chemical process or a biological process comprising:

(a) a reactor,

(b) a device for sampling the process,

(c) an interface,

(d) an isotope analyzer, and

(e) a computerized data system (CDS).

In another aspect the present invention relates to a system wherein the device for sampling the process samples the process at a selected frequency to obtain one or more samples from the process.

In another aspect the present invention relates to a system wherein the isotope analyzer is used to determine the isotopic ratio information for one or more isotope ratios from elements present in each sample.

In another aspect the present invention relates to a system wherein the isotope analyzer is selected from a cavity ring-down spectrometer (CRDS), an isotope-ratio mass spectrometer (IRMS), or a nuclear magnetic resonance (NMR) spectrometer.

In another aspect the present invention relates to a system wherein the isotope analyzer is a cavity ring-down spectrometer (CRDS).

In another aspect the present invention relates to a system wherein the isotope analyzer is an isotope-ratio mass spectrometer (IRMS).

In another aspect the present invention relates to a system wherein the isotope analyzer is a nuclear magnetic resonance (NMR) spectrometer.

In another aspect the present invention relates to a system wherein the computerized data system (CDS) is used for collecting and analyzing or processing the output from the isotope analyzer.

In another aspect the present invention relates to a system wherein the computerized data system further stores and displays the output analyzed or processed from the isotope analyzer.

In another aspect the present invention relates to a system further comprising a feedback loop operably connected to the computer data system to adjust process parameters in the process using defined routines if the isotopic information is outside acceptable ranges.

In another aspect the present invention relates to a system wherein the chemical process or the biological process is a chemical process.

In another aspect the present invention relates to a system wherein the chemical process is a chemical reaction.

In another aspect the present invention relates to a system wherein the chemical reaction is a batch chemical reaction.

In another aspect the present invention relates to a system wherein the chemical reaction is a continuous chemical reaction.

In another aspect the present invention relates to a system wherein the continuous chemical reaction is a flow chemical reaction.

In another aspect the present invention relates to a system wherein the chemical process or the chemical reaction is utilized for the manufacture of a pharmaceutical product.

In another aspect the present invention relates to a system wherein the chemical process or the biological process is a biological process.

In another aspect the present invention relates to a system wherein the biological process is a biological reaction.

In another aspect the present invention relates to a system wherein the biological reaction is a batch biological reaction.

In another aspect the present invention relates to a system wherein the biological reaction is a continuous biological reaction.

In another aspect the present invention relates to a system wherein the biological reaction is a flow biological reaction.

In another aspect the present invention relates to a system according wherein the biological process or the biological reaction is utilized for the manufacture of a biological product.

In another aspect the present invention relates to a system wherein the process is a biological process such as a fermentation process.

In another aspect the present invention relates to a system wherein the fermentation process is a beer fermentation process.

In another aspect the present invention relates to a system wherein the elements are selected from elements that have two or more isotopes.

In another aspect the present invention relates to a system wherein the elements are selected from elements that have two or more stable isotopes.

In another aspect the present invention relates to a system wherein the elements are selected from elements that have two or more naturally-occurring stable isotopes.

In another aspect the present invention relates to a system wherein the elements are selected from hydrogen, carbon, nitrogen, oxygen, sulfur, chlorine, bromine, and combinations thereof.

In another aspect the present invention relates to a system wherein the isotopes are stable isotopes.

In another aspect the present invention relates to a system wherein the isotopes are naturally-occurring stable isotopes.

In another aspect the present invention relates to a system where the isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In another aspect the present invention relates to a system wherein the isotope ratios are selected from the following pairs of isotopes: ¹H and ²H, ¹²C and ¹³C, ¹⁴N and ¹⁵N, ¹⁶O, and ¹⁸O, ³²S and ³⁴S, and ³⁵Cl and ³⁷Cl, and ⁷⁹Br, and ⁸¹Br, and combinations thereof.

In another aspect the present invention relates to a system wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br, and combinations thereof.

In another aspect the present invention relates to a system wherein the isotope ratio is ²H/¹H.

In another aspect the present invention relates to a system wherein the isotope ratio is ¹³C/¹²C.

In another aspect the present invention relates to a system wherein the isotope ratio is ¹⁵N/¹⁴N.

In another aspect the present invention relates to a system wherein the isotope ratio is ¹⁸O/¹⁶O.

In another aspect the present invention relates to a system wherein the isotope ratio is ³⁴S/³²S.

In another aspect the present invention relates to a system wherein the isotope ratio is ³⁷Cl/³⁵Cl.

In another aspect the present invention relates to a system wherein the isotope ratio is ⁸¹Br/⁷⁹Br.

In another aspect the present invention relates to a system wherein the isotopic information is intrinsic isotopic information.

In another aspect the present invention relates to a system wherein the isotopic information is obtained from CO₂ or CO produced from the process.

In another aspect the present invention relates to a system wherein the isotopic information is obtained from CO₂ produced by the process

In another aspect the present invention relates to a system wherein the isotopic information is obtained from CO produced by the process.

In another aspect the present invention relates to a system wherein the isotopic information is obtained from the isotope ratio of the ¹³C/¹²C or the ratio of ¹⁸O/¹⁶O, or combinations of these ratios from CO₂ or CO produced from the process.

In another aspect the present invention relates to a system wherein the isotopic information is obtained from the isotope ratio of the ¹³C/¹²C from the CO₂ or CO produced from the process.

In another aspect the present invention relates to a system wherein the isotopic information is obtained from the isotope ratio of the ¹³C/¹²C from the CO₂ produced from the process.

In another aspect the present invention relates to a system wherein the isotopic information is obtained from the isotope ratio of the ¹³C/¹²C from the CO produced from the process.

In another aspect the present invention relates to a system wherein the isotopic information is obtained from the isotope ratio of the ¹⁸O/¹⁶O from the CO₂ or CO produced from the process.

In another aspect the present invention relates to a system wherein the isotopic information is obtained from the isotope ratio of the ¹⁸O/¹⁶O from the CO₂ produced from the process.

In another aspect the present invention relates to a system wherein the isotopic information is obtained from the isotope ratio of the ¹⁸O/¹⁶O from the CO produced from the process.

In another aspect the present invention relates to a method for continuously monitoring the progress of a fermentation process which produces one or more products or byproducts selected from a byproduct such as gaseous CO₂, a byproduct such as pyruvic acid, a product such as ethanol, and mixtures thereof, comprising the steps of:
(a) continuously sampling one or more of the products or byproducts from the process,
(b) continuously determining or assessing the isotopic information for one or more isotope ratios from elements present in each sample or from the continuous sampling, wherein the isotope ratios are selected from ²H/¹H, ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

In another aspect the present invention relates to a method for continuously monitoring the progress of a fermentation process wherein the byproduct is CO₂ and the isotope ratio is selected from ¹³C/¹²C, ¹⁸O/¹⁶O, and combinations thereof.

In another aspect the present invention relates to a method for continuously monitoring the progress of a fermentation process wherein the byproduct is pyruvic acid and the isotope ratio is selected from ²H/¹H, ¹³C/¹²C, ¹⁸O/¹⁶O, and combinations thereof.

In another aspect the present invention relates to a method for continuously monitoring the progress of a fermentation process wherein the product is ethanol and the isotope ratio is selected from ²H/¹H, ¹³C/¹²C, ¹⁸O/¹⁶O, and combinations thereof.

In another aspect the present invention relates to a method for continuously monitoring the progress of a fermentation process wherein the isotope ratio is ¹³C/¹²C.

In another aspect the present invention relates to a method for continuously monitoring the progress of a fermentation process wherein the fermentation process is a beer fermentation process.

### DEFINITIONS

As used herein, the following terms have the following meanings unless expressly stated to the contrary:

The term “batch” as used herein refers to a process involving a quantity of material prepared or required for one operation or step, or the quantity produced at one operation. In a batch process, the output of that process can be passed to a subsequent process for further processing. A “batch process” or “batch processing” is in contrast to a “continuous process” or “continuous processing”.

The term “biological product” or “biologic product” as used herein refers to a biologically-produced medical product, which is commonly referred to as a “biologic”. Examples of biological products include medicinal products such as vaccines, blood, blood components, antibodies such as monoclonal antibodies, enzymes, proteins, and the like. Biological products also include materials for viral gene therapy for artificially manipulating a virus to include a desired piece of genetic material into a target gene or cell. In general, biological products are produced by biological processes, such as e.g., fermentation, cell cultures, extractions, purifications, and harvesting from biological sources. Biological products are also produced by genetic engineering techniques such as, e.g., recombinant DNA and RNA procedures, polymerase chain reaction (PCR) amplification, and the like. Biological products are also produced by derivatization and modification of natural product sources. Biological products generally are made by biological processes rather than chemical processes.

The term “continuous” as used herein refers to a “continuous process” and also a method or system for “continuously monitoring” a process, “continuously sampling a process”, and “continuously determining” (with respect to the process) whether it is a continuous process or a batch process. A “continuous process” is one that is designed to run non-stop. A “continuous process” or “continuous processing” is in contrast to a “batch process” or “batch processing”. “Continuously monitoring” means that the methods and systems are such that they sample, monitor, measure, or determine (i.e. make determinations with respect to the process) the processes of the present invention down to very small time intervals, such that the sampling, monitoring, measuring, or determining, for all intents and practical purposes, is essentially instantaneous. Such sampling, monitoring, measuring, or determining can then be conducted at one or more time points or at desired time intervals. Alternatively, the sampling, monitoring, measuring, or determining is made from a gaseous stream or outflow, or from a liquid stream or outflow from the chemical process or the biological process, a non-limiting example of such being wherein an inert gas, such as helium, is continuously run over or through the chemical or biological process to continuously sweep out or remove a gaseous product or by-product, such as CO₂ or CO. The isotopic information is continuously determined on the gaseous product or by-product to monitor the progress of the chemical process or the biological process. In the foregoing described in this paragraph, the output of such sampling, monitoring, measuring, or determining is essentially continuous.

The term “first time point” as used herein refers to the first point in time or the initial point in time at which the process is sampled and the first or initial isotope information is determined or assessed. The term “first time point” is synonymous with “initial time point”. The term “first time point” or “initial time point” is intended to be distinguished from one or more subsequent time points or later time points, at which the process is sampled and subsequent or later isotope information is determined or assessed.

The term “flow” as used herein refers to a continuous chemical or biological process wherein the feedstocks, starting materials or reactants are provided in a flow or stream and the desired product or products are removed as an effluent flow or stream.

The term “incremental” as used herein refers to an additional increase in quantity, and in most cases a small or minute, but measureable, increase in quantity. The term as used herein refers to an incremental yield for a chemical or biological process.

The term “instantaneous” as used herein refers to something that happens or occurs very quickly or in an instant, or in other words, in a very small, but measureable increment of time. The term “instantaneous” as used herein also refers to an instantaneous yield for a chemical or biological process. Because it is recognized that the methods and systems of the present invention may not strictly provide instantaneous sampling, monitoring, or measuring, the term “instantaneous” is also meant to include the terms “substantially instantaneous” and “essentially instantaneous”, to convey the concept that for all intents and practical purposes these methods and systems are instantaneous.

The term “process” as used herein refers to one or more actions or operations for making, producing or manufacturing a product. The term is intended to include chemical processes and biological processes. The term process is also is also intended to include the sum of one or more reactions, which can be chemical reactions or biological reactions. The processes and reactions include feedstocks, starting materials, reactants, solvents, catalysts; physical parameters such as temperature, pressure, agitation, atmospheric conditions, aeration, and gas through-put; and time variables; and the like.

The terms “reaction or reactions” as used herein refer to the chemical or biological reactions of the processes of the present invention. A reaction is generally a discrete chemical or biological step or transformation.

The term “stable isotope” or “stable isotopes” as used herein refers to those isotopes that have never been observed to decay. It is recognized that all isotopes will eventually decay. Some isotopes such as hydrogen-7 (⁷H) and lithium-4 (⁴Li) have half-lives on the order of 10⁻²⁴ seconds, whereas, in contrast, calcium-48 (⁴⁸Ca) and tellurium-148 (¹⁴⁸Te) have half-lives on the order of 10²⁴ years. The stable isotopes useful in the present invention are generally the naturally-occurring stable isotopes of hydrogen, carbon, nitrogen, oxygen, sulfur, chlorine, and bromine. More specifically these naturally-occurring stable isotopes are hydrogen (hydrogen-1 or ¹H), deuterium (hydrogen-2 or ²H), carbon-12 (¹²C), carbon 13 (¹³C), nitrogen-14 (¹⁴N), nitrogen-15 (¹⁵N), oxygen-16 (¹⁶O), oxygen-18 (¹⁸O), sulfur-32 (³²S), sulfur-34 (³⁴S), chlorine-35 (³⁵Cl), chlorine-37 (³⁷Cl), bromine-79 (⁷⁹Br), and bromine-81 (⁸¹Br).

The term “stream” as used herein refers to a continuous flow process wherein either the continuous feedstock, starting materials, or reactants are conveyed to or flow to the process or the reactor or vessel in which the process occurs; or wherein the effluent containing the desired product or products is removed from or where conveyed from, or flows from the process or the reactor or vessel in which the process occurs. The process can be considered a flow or stream process.

The symbol, δ, is a measure of isotopic abundance, and δ is usually reported as the difference in parts per thousand, or permil (‰), from an international standard. δ can be negative or positive depending on whether the sample is enriched or depleted in the heavy isotope relative to a standard.

The symbol, ‰, designates permil or what is also referred to as parts per thousand.

Stable Isotope Identification Methods

Measurements of the abundances of naturally occurring stable isotopes in pharmaceutical materials can be used to quantitatively characterize both the sources of the products and the synthetic processes used to produce them, as well as the progress of those processes. The methods and systems of the present invention utilize isotopic information for one or more isotope ratios from elements present in samples from the chemical or biological processes of interest. Methods and systems for isotope identification are described in U.S. Pat. No. 7,323,341 B1, to Jasper, issued Jan. 29, 2008; U.S. Pat. No. 8,367,414 B2, to Jasper, issued Feb. 5, 201; and J. P. Jasper, L. E. Weaner, and J. M. Hayes, Process Patent Protection: Characterizing Synthetic Pathways by Stable-Isotope Measurements, Pharmaceutical Technology, 2007, 31(3):68-73; which are incorporated by reference herein in their entirety.

For many products, e.g. such as a pharmaceutical product, the source of each atom is known in detail. For example, a methyl carbon will derive from a particular synthetic reactant, an amino nitrogen from another, etc. The measured carbon or nitrogen isotopic composition of the final product will be the weighted average of all carbon or nitrogen positions within the molecule. In turn, this measured isotopic composition will be equal to the weighted average of the isotopic compositions at the precursor positions in the synthetic reacts as modified by generally only two factors: (i) if the synthetic reactions are non-quantitative, any isotope effects which modulate the transfer of material from reactant to products and (ii) in some cases, exchanges of isotopes between products and reaction media.

Isotopic calculations are based on two systems of equations. The first employs mass balances and the second involves integrated forms of rate equations that pertain to kinetically controlled isotopic fractionations. Equations describing mass balances are generally exact when cast in terms of fractional abundances [e.g., ¹³C/(¹²C+¹³C)]. In contrast, assessments of differential rates are based on isotope ratios (e.g., ¹³C/¹²C). When these systems are blended, either approximations or equations with multiple terms are employed. For details, see Hayes J M, 2004; ttp://www.nosmas.whoi.edu/docs/IsoCalcs.pdf, which is incorporated by reference herein in its entirety.

The relevant isotopic parameters are stoichiometry (n), isotopic abundance (δ), the magnitude of the isotopic effect (ε), and a variable related to conversion of reactants to products (f).

The symbol, n represents the stoichiometry of the reaction, more specifically the number of atoms of a given element (e.g., carbon) in a given molecule involved in the reaction.

As mentioned above, (δ), is a measure of isotopic abundance, and δ is usually reported as the difference in parts per thousand, or permil (‰), from an international standard. δ can be negative or positive depending on whether the sample is enriched or depleted in the heavy isotope relative to the standard. For example, in the case of carbon the difference is calculated as

δ¹³C(‰)=([(Rₛₘₚₗ)/(R_{std})]−1)·(1000) (equation 1)

where Rₛₘₚₗ is the ¹³C/¹²C ratio of the sample and R_{std} is the ¹³C/¹²C ratio in the standard. δ is thus linearly proportional to the isotopic ratio in the sample. Standards are available from the International Atomic Energy Authority and a standard for each isotope is used to determine the zero point of an abundance scale for that isotope. Standards include a particular seawater sample for H and O, calcium carbonate for C, air for N, and a meteorite for S. When the sample is depleted in the heavy isotope relative to the standard, δ is negative and when the sample is enriched it has a positive value. If it has the same isotopic abundance then δ=0. See, J P Jasper, The Increasing Use of Stable Isotopes in the Pharmaceutical Industry, Pharm. Tech., 1999, 23(10):106-114, which is incorporated by reference herein in its entirety.

The magnitude of an isotope effect, (e), is such that its value depends on details of the reaction and on the relative mass difference between isotopes. Effects are largest for D (deuterium) vs. H and smaller for heavier elements. In general, the values of ε are specific to individual positions within the molecules involved. The isotope effects are largest at the reaction site, much smaller at neighboring positions, and usually not measurable elsewhere. Like δ, ε relates to the isotopic difference between two materials (e. g., reactant and product) and is usually expressed in permil or parts per thousand. For example, for kinetic isotope effects, in the methods and systems employed here, ε=−10‰ means that a reaction site bearing the heavy isotope reacts 10 parts per thousand, or 1%, more slowly than a site bearing a light isotope. For equilibrium isotope effects, ε_{A/B}=15‰ would mean that, at equilibrium, A Is enriched in the heavy isotope by 15 parts per thousand relative to B. Here, A and B refer to specific atomic positions that can be related by a chemical equilibrium.

f is a measure of the progress of a reaction. It is generally the most important variable governing fractionations caused by isotope effects. Its value ranges from 1 to 0 and depends on factors such as temperature, pressure, or availability of reactants. In equilibria (A⇄B), f indicates the position of the equilibrium, with f_{B}=1 indicating complete conversion to B and, at any position, f_{A}+f_{B}=1. In irreversible reactions, f_{X} indicates the portion of reactant X which remains unconsumed, with f_{X}→0 as the reaction proceeds to completion.

The precision of isotopic analyses is typically calculated by two methods. Pooled standard deviations of raw data are typically computed from sets of duplicate or triplicate measurements. From those pooled standard deviations, standard deviations of mean values pertaining to specific substances are calculated. More specifically, the standard deviation of a mean value is the pooled standard deviation divided by n^{1/2}, where n is the number of measurements performed on a given sample. See Jasper, J P, Quantitative estimates of precision for molecular isotopic measurements. Rap. Comm. Mass Spec., 2001 15:1554-1557, which is incorporated by reference herein in its entirety. For carbon, nitrogen, oxygen, and sulfur, the resulting 95% confidence intervals for a result are typically in the range of ±0.1- to ±0.4‰. For hydrogen, the 95% confidence interval is typically ±3‰.

Precise quantitation of stable isotopic compositions in pharmaceutical intermediates and products requires both mass balance and isotopic fractionation equations that are applicable to both single and multi-step reaction sequences. One starts from the most basic requirement of mass balance then considers isotopic fractionations in a single reaction.

Mass Balance

For A+B→C, where reactants A and B are quantitatively converted to product C, two mass balances can be written:

m_{A}+m_{B}=m_{C} equation (2)

m_{A}δ_{A}+m_{B}δ_{B}=m_{C}δ_{C} equation (3)

where, m_{A}, m_{B}, and m_{C} are molar amounts of carbon (or any other element) in A, B, and C and the isotopic compositions of that carbon (or any other element) in A, B, and C are given by δ_{A}, δ_{B}, and δ_{C}. Equation 2 is a mass balance (i.e., carbon in=carbon out) while equation 3 is an isotopic mass balance (¹³C in=¹³C out). Under the conditions postulated (quantitative conversion) the isotopic composition of C can be computed from those of A and B. See, Hayes J M, 2004; http://www.nosmas.whoi.edu/docs/IsoCalcs.pdf and J P Jasper, The Increasing Use of Stable Isotopes in the Pharmaceutical Industry, Pharm. Tech., 1999, 23(10):106-114, which are incorporated by reference herein in their entirety.

Isotopic Fractionation

For isotopic fractionations, calculations should take into account factors such as reaction completeness and isotope effects, as these will cause the isotopic composition of C to differ from that computed using the mass balance equation and assuming quantitative conversion of reactants to products. To provide a concrete example, assume that A is present in excess while B, the limiting reactant, is quantitatively converted to product. In that case

n_{A}(δ_{A}−Δ_{A})+n_{B}δ_{B}=n_{C}δ_{C} equation (4)

where n_{A}, n_{B}, and n_{C} represent the numbers of atoms of carbon (or any other element of interest) in A, B, and C. Because A is not quantitatively converted to product, the isotopic compositions of the A-derived positions in C can differ from those in the initial reactant. Here, that isotopic offset, or change, is expressed as Δ_{A}, where its value depends on the isotope effect(s) and on the fraction of A that remains unconsumed. If the reaction conditions, particularly the magnitude of the excess of A, are consistent, Δ_{A} will be constant. Because the n values are known exactly, Δ_{A} can be determined from equation 4 after isotopic analysis of the reactants and product (i.e., determination of δ_{A}, δ_{B}, and δ_{C}).

Values of δ_{A}, δ_{B}, and δ_{C} generally do not affect the values of Δ_{A}. Accordingly, once Δ_{A} is known for a given reaction and set of conditions, it is usually necessary only to know two of the δ values in order to compute the third. Thus, for example, when δ_{A}, δ_{A}, and δ_{B}, are known, the isotopic value of the product (δ_{C}) can be calculated.

If neither A nor B is completely consumed during the course of the reaction, and if the rate of the chemical reaction (or position of the chemical equilibrium) is sensitive to isotopic substitution on both reactants, it will be necessary to consider values of the offset, or change, of both Δ_{A} and Δ_{B}:

n_{A}(δ_{A}−Δ_{A})+n_{B}(δ_{B}−Δ_{B})=n_{C}δ_{C} equation (5)

If reaction conditions cannot be manipulated so that f_{A} and f_{B} (and thus Δ_{A} and Δ_{B}) can be independently driven to completion (i.e., zero), it will be possible to determine only the sum, n_{A}δ_{A}+n_{B}δ_{B}. From theoretical considerations, Δ_{A} and Δ_{B} can be evaluated separately for all values of f_{A} and f_{B} if the isotope effects are known. See, Scott, K M, Lu, X, Cavanaugh, C M, and Liu, J S, Geochim. Cosmochim. Acta, 2004; 68(3):433, which is incorporated by reference herein in its entirety.

Of course, isotopic fractionations like those discussed above accumulate during the different steps of a multi-step synthesis scheme. They can, however, be individually and systematically differentiated, not only for multiple reactants but also for multiple isotopes. To provide an example consider carbon-isotopic fractionations in a hypothetical four-step sequence:

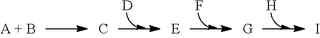

Illustrative carbon skeletons for reactants and products are shown below with pertinent quantities summarized in Table 1.

Illustrative Carbon Skeletons for Reactants and Products

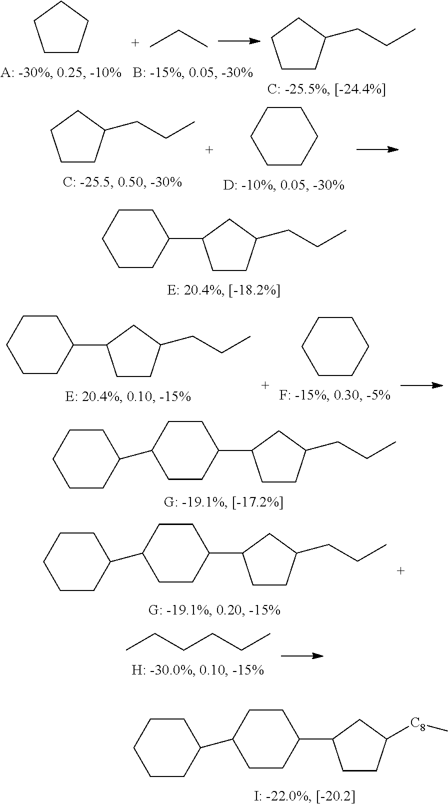

Carbon skeletons of reactants and products in a hypothetical four-step synthetic reaction scheme. This example illustrates the effects of the four key isotopic variables (n, δ, f, ε) on the isotopic compositions of the three synthetic intermediates (C, E, G) and of the final product, I (δ_{P}). For all eight reactants (left), the given numerical values are δ, f, and ε. For all four products (right), the numerical values are the isotopic compositions actually observed (δ_{P}) and that expected in the absence of isotope effects and incomplete consumption of reactants ([δ_{P}*]).

TABLE 1
Properties of Four-Step Synthetic Sequence^{a}

| Reactants | | | | | | Conditions | | | | Products | | |
|---|---|---|---|---|---|---|---|---|---|---|---|---|
| 1 | 2 | n₁ | n₂ | δ₁, ‰ | δ₂, ‰ | f₁ | f₂ | Σε₁, ‰ | Σε₂, ‰ | | δ_{P}*, ‰ | δ_{P}, ‰ |
| A | B | 5 | 3 | −30.0 | −15.0 | 0.255 | 0.055 | −10.0 | −30.0 | C | −24.4 | −25.5 |
| C | D | 8 | 6 | −25.5 | −10.0 | 0.50 | 0.05 | −30.0 | −5.0 | E | −18.2 | −20.4 |
| E | F | 14 | 6 | −20.4 | −15.0 | 0.10 | 0.30 | −15.0 | −5.0 | G | −17.2 | −19.1 |
| G | H | 20 | 6 | −19.1 | −30.0 | 0.20 | 0.10 | −15.0 | −15.0 | I | −20.2 | −22.0 |

^{a}The sequence of reactants and products is given by equation 6 in the text. The numbers of carbon atoms and the carbon-isotopic compositions of reactants 1 and 2 in each step are given by n₁, n₂, δ₁, and δ₂. The fractions of each reactant unconsumed in each step are given by f₁ and f₂. The sums of all carbon isotope effects pertaining to each reactant are given by Σε₁ and Σε₂. The isotopic compositions that successive products would have in the absence of isotope effects are given by δ_{P}* and the isotopic compositions actually observed are given by δ_{P}.

The carbon numbers (n₁, n₂), initial isotopic compositions (δ₁, δ₂), fractions of reactants remaining unconsumed (f₁, f₂) and summed isotope effects (Σε₁, Σε₂) are chosen to be representative of a typical synthetic scheme. All isotope effects are assumed to be kinetic. Values of δ_{P}*, the isotopic compositions that would be observed if isotopic fractionations were absent, are calculated using equation 5 with Δ_{A}=Δ_{B}=0; that is, the simple mass balance equations 2-3. Values of δ_{P}, the isotopic compositions that would actually be observed for the successive products, are calculated using exact forms of integrated rate equations. See Scott, K M, Lu, X, Cavanaugh, C M, and Liu, J S, Geochim. Cosmochim. Acta, 2004; 68(3):433, which is incorporated by reference herein in its entirety.

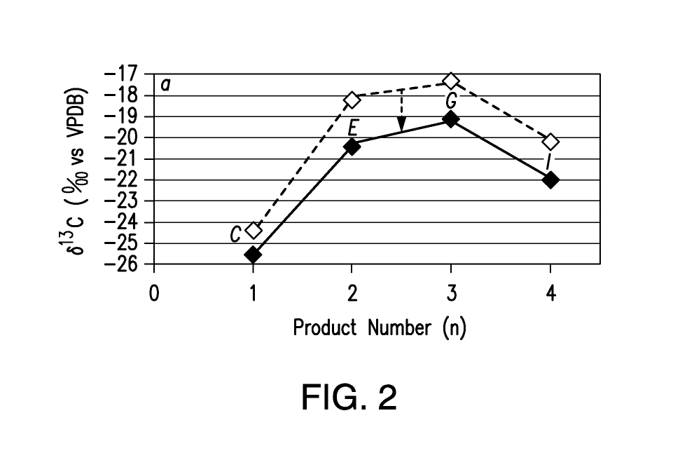

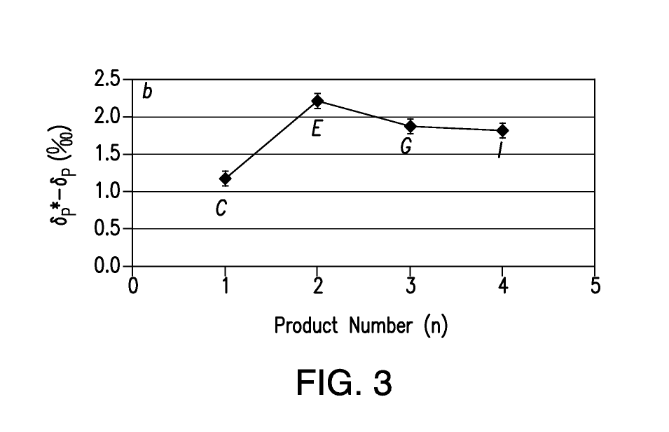

The foregoing illustrates the interplay of the four factors that control the isotopic compositions of manufactured products, namely the stoichiometries and isotopic compositions of the starting materials, isotope effects associated with the synthetic reactions, and the degree to which conversions of precursors to products are quantitative. The isotopic compositions of products are generally dominated by the initial isotopic abundance of the precursor materials and are variously modulated (viz., depleted) by the degree of completion (f) and the magnitude of any isotopic effects (ε, FIG. 2). A plot that summarizes the difference between the isotopic compositions that are predicted and those that would be observed in the absence of isotope effects (δ_{P}*−δ_{P}) is shown in FIG. 3. These values are also shown in the last two columns of Table 1. In the first synthetic step, isotope effects on reactant B are rather large, but that reactant is consumed almost completely. The resulting isotopic fractionation is less than 1‰ (the larger value shown in FIG. 3 pertains to the product and reflects fractions affecting both reactants). In the second step, a large isotope effect and poor conversion of reactant C lead to a large isotopic fractionation at the reaction site. However, fractionation is diluted now that the product contains 14 carbon atoms. As shown in FIG. 3, the overall difference between real and hypothetical unfractionated products is barely doubled. In the remaining steps, where isotope effects are moderate and consumption of reactants is relatively efficient, isotopic generally fractionation declines.

For a chemical or biological process, one can monitor the equilibrium between two isotopes, which are designated as “A” and “B”. Consider the case of a general system in which there are three different atoms or isotopes under consideration:

A+B→P

where A, B, and P contain n_{A}, n_{B}, and n_{P} atoms of the element under consideration. The system can be described by the following equation for determining the progress of the process via the isotopic abundance, δ:

$${δ}_{P} = {δ}_{P}^{*} - \frac{1}{{n}_{P}} [ \frac{{f}_{A}}{1 - {f}_{A}} ln {f}_{A} ( {ɛ}_{A 1} + {ɛ}_{A 2} + … ) + \frac{{f}_{B}}{1 - {f}_{B}} ln {f}_{B} ( {ɛ}_{B 1} + {ɛ}_{B 2} + … ) ] \qquad equation ( 6 )$$

In equation 6, δ_{P} and δ_{P}* are the observed isotopic composition of the product and the idealized isotopic composition of the product, respectively, ε_{A1}, ε_{B2}, etc. are the primary kinetic isotope effects at the reaction sites in A and B, respectively, and ε_{A2}, ε_{B2}, etc. are the secondary isotope effects, and f is a measure of the progress of the reaction.

Continuous Monitoring Methods

The present invention relates to methods for continuously monitoring the progress of chemical or biological processes utilizing isotopic information for one or more isotope ratios from elements present in samples from these processes. These continuous methods utilize the stable isotope identification methods as described herein.

Until recently, it was generally not possible to sample, monitor, or measure a process for isotopic determinations more frequently than say, once about every 15 minutes, and thus not possible to provide methods and systems for continuous isotope information determination. The reason for these relatively long time intervals was due to sampling and instrumentation limitations. In the present invention, it is now possible to sample, monitor, or measure at or over very small time intervals, the result which is for all intents and practical purposes, is perceived as a continuous sampling, monitoring, or measuring of the chemical or biological process.

Continuous Monitoring System

The present invention relates to systems for continuously monitoring the progress of chemical or biological processes utilizing isotopic information for one or more isotope ratios from elements present in samples from these processes. The systems of the present invention are useful for carrying out the methods of the present invention. The systems of the present invention comprise the following components, each of which are described in further detail: (a) a device for sampling the process, (b) an interface, (c) an isotope analyzer, and (d) a computerized data system (CDS). In further embodiments, the systems of the present invention can also include a reactor in which the chemical processes or the biological processes of the present invention are conducted or contained.

Device for Sampling the Process

The systems of the present invention comprise a device for sampling the process.

This device is an instrument or probe that samples materials from the reaction system or vessel. The device can also be a stream of an inert gas which is either bubbled through or passed over the system for removing, i.e. sampling gaseous or volatile products or by-products of the chemical or biological process. An example of such a sampling system can be a tube, hose, or line for delivering a stream of an inert gas, such as helium, and a corresponding tube, hose, or line for collecting the effluent gas and any desired products or by-products for isotope analysis.

Interface

The systems of the present invention comprise an interface.

The interface is the connector between the sampling device and the isotope analyzer. This interface can take a variety of forms and can be either electronic or mechanical.

Isotope Analyzer

The systems of the present invention comprise an isotope analyzer.

In theory, a wide range of isotope analyzers can be used in the systems of the present invention. The isotope analyzer is a device for measuring or determining the desired stable isotope ratios of the sampled process. Examples of isotope analyzers useful for the methods and systems of the present invention include those selected from: (a) cavity ring-down spectrometer (CRDS), (b) an isotope ratio mass spectrometer (IRMS), and (c) a nuclear magnetic resonance (nmr) spectrometer.

Cavity Ring-Down Spectrometer (CRDS)

Cavity ring-down spectroscopy (CRDS) is an optical spectroscopic technique utilizing a cavity ring-down spectrometer (CRDS). The method is highly sensitive, down to the 0.1% level, and is used to measure the light absorption of samples, i.e. the absolute optical extinction, that scatter and absorb light such as gas samples. A common cavity ring-down spectrometer configuration comprises a laser used to illuminate a high-finesse optical cavity, which essentially comprises two highly reflective mirrors. When the laser is in resonance with a cavity mode, the intensity of the laser light builds up in the cavity due to constructive interference. When the laser is turned off, the exponentially decaying light intensity leaking from the cavity is measured. This decaying laser light is reflected between the mirrors many thousands of time giving an effective path length on the order of kilometers.

When a sample is placed in the cavity, such as a sample containing a desired isotope, the intensity of the light decreases faster due to the absorption of the sample. The cavity ring-down spectrometer measures how long it takes for the light to decay, or “ring-down” to lie of its initial intensity both with and without the sample, thus giving a measure of the amount of the sample absorbing the laser light. See, Giel Berden; Rudy Peeters; Gerard Meijer (2000). “Cavity ring-down spectroscopy: Experimental schemes and applications”. International Reviews in Physical Chemistry 19 (4): 565-607; and Paldus, B. A. and Kachanov, A. A., An Historical Overview of Cavity Enhanced Methods (Einstein Centennial Review Article), Canadian Journal of Physics, 83, pp. 975-999 2005 NRC; which are incorporated by reference herein in their entirety.

An example of a cavity ring-down spectrometer useful in the methods and systems of the present invention includes a Picarro CRDS G2131-i Analyzer sold by Picarro Inc., 3105 Patrick Henry Drive, Santa Clara, Calif. 95054.

Isotope Ratio Mass Spectrometer (IRMS)

Isotope-ratio mass spectrometry (IRMS) is a type of mass spectrometry. The method uses an isotope-ratio mass spectrometer (IRMS) measure the relative abundance of isotopes in a given sample. For the methods and systems of the present invention, isotope-ratio mass spectrometry is used to measure or analyze the isotopic variations of stable isotopes in samples of interest. The isotope-ratio mass spectrometer (IRMS) allows the precise measurement of mixtures of naturally occurring isotopes. See, Townsend, A. (ed) (1995) Encyclopaedia of Analytical Science Encyclopaedia of Analytical Science. London: Academic Press Limited, which is incorporated by reference herein in its entirety.

Isotope-ratio mass spectrometers useful herein can be of either the magnetic sector design or the quadrupole design, with the magnetic sector design generally being preferable. The magnetic sector type, also known as the “Nier type”, after its designer Alred Nier, operates by ionizing the sample and accelerating it over a potential (usually in the kilo-volt range). The resulting stream of ions is thus separated according to their mass-to-charge ration, or m/z.

See, Goetz, A.; Platzner, I. T. (Itzhak Thomas); Habfast, K.; Walder, A. J. (1997). Modern isotope ratio mass spectrometry. London: J. Wiley, which is incorporated by reference herein in its entirety.

An example of an isotope-ratio mass spectrometer useful herein is a ThermoScientific DELTA V™ Plus Isotope Ratio Mass Spectrometer. See, http://www.thermoscientific.com/en/product/delta-v-plus-isotope-ratio-mass-spectrometer.html, which is incorporated by reference herein in its entirety.

Nuclear Magnetic Resonance (NMR) Spectrometer

A nuclear resonance (NMR) spectrometer is a very common analytical device that is even now available in many undergraduate chemistry laboratories. NMR spectroscopy is an analytical method that uses the magnetic properties of certain atomic nuclei to provide both qualitative and quantitative physical and chemical properties of atoms and the molecules in which they are contained. When placed in a magnetic field, various nuclei or isotopes, e.g., ¹H and ¹³C, absorb electromagnetic radiation at a frequency characteristic of the isotope. Such information can include structures, dynamics, chemical environment, and also isotope and isotope ratio information.

See V. Govindaraju, K. Young, and A. A. Maudsley, Proton NMR chemical shifts and coupling constant for brain metabolites. NMR in Biomedicine, Volume 13, Issue, pages 129-153, May 2000; and J. H. H. Nelson and J. H. Nelson, Nucelar Magnetic Resonance Spectroscopy: 1^{st} Edition, ISBN-13: 9780130334510, 2002, Prentice Hall, which are incorporated by reference herein in their entirety.

An example of a nuclear magnetic resonance spectrometer useful herein is a Thermo Scientific picoSpin 80 NMR Spectrometer.

Computerized Data System (CDS)

The systems of the present invention comprise a computerized data system (CDS). A computerized data system is the computer or computer system for collecting, processing, and storing the isotope ratio data generated from the sampling and collection of samples from the processes of the present invention. In many cases, the computerized data system is integrated into or closer associated with the isotope analyzer. In other others it is a separate or stand-alone computer, whether a hand-held, lap-top, desk-top, or main-frame computer which is attached or associated with the isotope analyzer. By associated is meant that the data from the isotope analyzer is either sent electronically, wirelessly, or transmitted via a separate storage device such as a CD or flash-drive.

Reactor

The processes described herein, whether chemical or biological, are generally conducted in some type of reactor or vessel. Although not strictly a component of the systems of the present invention, the reactor can in some embodiments be considered a component. In such cases, the systems of the present invention further comprise a reactor.

Chemical and biological reactors come in a wide array of forms varying from small size laboratory glassware such as test tubes and flasks, to scale-up and pilot plant systems, to large scale manufacturing plants. The reactor can be used to conduct a discrete or single batch process or reaction. Alternatively, the reactor can be one that operates on a continuous basis wherein a feedstock of starting materials or reactants are continuously supplied and a reaction effluent or product stream is continuously removed. Such continuous reactors can operate on a flow or stream basis.

The reactor can also be a fermentation vat or vessel, such as a beer fermentation vat, that is appropriately configured.

See R. Turton, R. C. Bailie, W. B. Whiting, J. A. Shaeiwita, and D. Bhattacharyya, Analysis, Synthesis and Design of Chemical Proceeses (4^{th} Edition) (Prentice Hall International Series in the Physical and Chemical Engineering Sciences), Jul. 2, 2012, which is incorporated by reference herein in its entirety.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### Method and System for Continuous Monitoring of Reaction Yield Via Online Stable-Isotope Ratio Monitoring Using a Cavity Ring-Down Spectrometer (CRDS)

Natural-abundance stable-isotope ratios are quantitatively related to the yield of reactions. See, J. P. Jasper, L. E. Weaner, and J. M. Hayes, Process Patent Protection: Characterizing Synthetic Pathways by Stable-Isotope Measurements, Pharmaceutical Technology, 2007, 31(3):68-73, which is incorporated by reference herein in its entirety. Until recently, the monitoring of such ratios was typically performed offline (e.g., J. P. Jasper, T. M. Schelhorn, and J. L. Treadway, A Stable Isotope Indirect Calorimeter for the Quantification of the Metabolic Rate of ¹³C-Labelled Metabolites in Mice, Abstract from The International Isotope Society, King of Prussia, Pa.: Oct. 27, 2000, which is incorporated by reference herein in its entirety. With the recent advent of continuous isotope-ratio monitoring via Cavity Ring-Down Spectroscopy we employ a Picarro CRDS G2131-i Analzer to monitor the isotopic composition (e.g., δ¹³C) as a quantitative index of yield.

For this continuous isotope ratio mass spectrometry experiment, real-time monitoring of reaction yield in production is a key parameter. For initial, independent calibration of yield, the integrated output of an online flow meter and an online pCO₂ (i.e. a partial pressure CO₂) meter (LI-800 CO2 Gas Hound, LI-COR Inc., Lincoln, Nebr., U.S.A.) permits a real-time estimate of the mass of CO₂ generated by the reactor up to the point of total yield. The present development of continuous, online isotope-ratio mass instruments presents an opportunity to monitor reaction yield in real time via the isotopic composition of the off gas from the reactor. We perform an experiment to illustrate the utility of natural-abundance stable isotopes in process chemistry. We use a reaction system that generates a product such as carbon dioxide, such as from a beer brewing system. Alternatively, we use a reaction system that generates carbon dioxide from a pharmaceutical manufacturing process, e.g., the removal of a BOC protecting group from a pharmaceutical product intermediate which has been protected with a BOC protecting group via di-tert-butyl dicarbonate (note that the BOC protecting group is generally used to protect amino groups). See, S. Doherty and A. Garrett, Another Tool from the PAT Tool Box, Spectrometry: Issue 4, Nov. 11, 2005, European Pharmaceutical Review, which is incorporated by reference herein in its entirety.

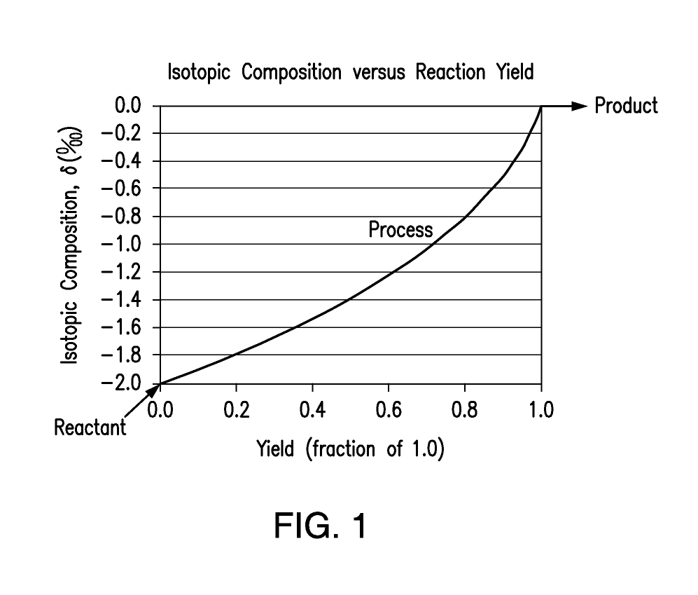

We employ a Picarro CRDS G2131-i Analyzer and a laptop computer to continuously monitor the δ¹³C—CO₂ generated in these reactions. FIG. 1 depicts the isotopic composition of such a reaction product plotted as a function of reaction yield. The isotopic composition, δ, increases as the reaction yield approaches 1, that is, as it approaches completion.

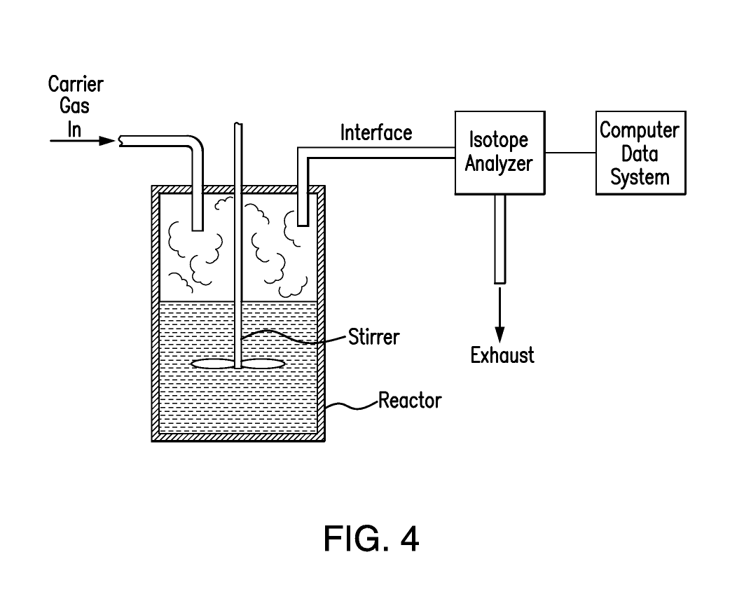

FIG. 4 depicts a system for continuously monitoring the progress of such a chemical process as per this Example 1, in which a gaseous product (or by-product, e.g., CO₂) is generated, e.g., the production of CO₂ from a fermentation process or a BOC deprotection reaction. This system illustrates a stirred reactor, a line for blowing a carrier gas (e.g., helium, nitrogen, or the like) through the system to continuous sample it to collect the gaseous product or by-product (e.g., CO₂), and an effluent tube which feeds in to an isotope analyzer and an associated computerized data system (CDS). The interface is essentially the connection of the effluent tube to the mass spectrometer. The progress of the fermentation is monitored via isotope information from the ¹³C/¹²C ratio of the CO₂ produced. Alternatively, the progress of the fermentation is monitored from the ¹⁸O/¹⁶O ratio of the CO₂ produced.

Alternatively, the method and system of Example 1 is used to monitor the progress of a reaction in the synthesis of a pharmaceutical product. In this case the progress of a BOC deprotection reaction of a pharmaceutical intermediate is monitored. The pharmaceutical intermediate is prepared via reaction of the desired precursor with a standard BOC reagent such as di-tert-butyl dicarbonate.

The progress of the deprotection reaction is monitored via the carbon dioxide that is liberated during the deprotection reaction by determining the ¹³C/¹²C ratio or the ¹⁸O/¹⁶O ratio of the CO₂ produced during the deprotection reaction.

The method and system described herein are useful for continuously monitoring the progress or reaction yield of a chemical or a biological process.

### Example 2

### Method and System for Continuous Monitoring of Reaction Yield Via Online Stable-Isotope Ratio Monitoring Using an Isotope Ratio Mass Spectrometer (IRMS)

The method and system of Example 2 is essentially the same as for Example 1, except that an Isotope Ratio Mass Spectrometer (IRMS), such as a ThermoScientific DELTA V™ Plus Isotope Ratio Mass Spectrometer, is employed in place of the cavity ring-down spectrometer (CRDS).

The method and system described herein are useful for continuously monitoring the progress or reaction yield of a chemical or a biological process.

### Example 3

### Method and System for Continuous Monitoring of Reaction Yield Via Online Stable-Isotope Ratio Monitoring Using a Nuclear Magnetic Resonance (NMR) Spectrometer

The method and system of Example 3 is essentially the same as for Example 1, except that a Nuclear Magnetic Resonance (NMR) Spectrometer, such as a Thermo Scientific picoSpin 80 NMR Spectrometer is employed in place of the cavity ring-down spectrometer (CRDS).

The method and system described herein are useful for continuously monitoring the progress or reaction yield of a chemical or a biological process.

### Example 4

### Determining the Efficiency of a Fermentation System by Isotope Ratio Monitoring

The efficiency of a fermentation system in generating product (P) and biomass (B) from a substrate (S) is determined by a stable-isotopic construction shown here. The basic reaction for this fermentation system is given by

S→P+B

and the mass balance for the substrate, product, and biomass is given by

φ_{S}=φ_{P}+φ_{B} (equation 7)

where,

φ_{S}=flux of substrate (S).

φ_{P}=flux of product (P); and,

φ_{B}=flux of biomass (B).

In this example the ¹³C/¹²C isotopic ratio is monitored, although other isotopic ratios can be used. The isotopic mass balance is given by

φ_{S}δ_{S}=φ_{P}δ_{P}+φ_{B}δ_{B} (equation 8)

where

δ_{S}=carbon-isotopic composition (δ¹³C) of substrate (S).

δ_{P}=carbon-isotopic composition (δ¹³C) of product (P); and,

δ_{B}=carbon-isotopic composition (δ¹³C) of biomass (B).

From (equation 7) and (equation 8), we calculate φ_{P}/φ_{S} and φ_{B}/φ_{S}:

φ_{P}/φ_{S}=(δ_{S}−δ_{B})/(δ_{P}−δ_{B}) (equation 9)

φ_{B}/φ_{S}=(δ_{S}−δ_{P})/(δ_{B}−δ_{P}) (equation 10).

where,

φ_{P}/φ_{S}=the flux ratio of product/substrate=efficiency of product production; and,

φ_{B}/φ_{S}=the flux ratio of biomass/substrate=efficiency of biomass production.

This monitoring analysis permits the opportunity to regulate the reaction system to optimize substrate utilization and product and biomass production.

This approach described in this example is more generally applicable to other reaction systems corresponding to the general scheme

S→P+B

where B is not necessarily restricted to a biomass and can be a second product or by-product, so long as one can isolate the three major components (S, P, and B) and isotopically analyze them. The method and system described herein are useful for continuously monitoring the fermentation efficiency of a process.

### Example 5

### Model Method and System for Continuous Monitoring of a Fermentation Process

A fermentation system such as a beer fermentation system is set up in which the CO₂ produced is continuously monitored. A system as is depicted in FIG. 4 can be used to conduct the fermentation process, as long as the resultant CO₂ is continuously monitored. In the case of beer fermentation, an appropriate fermentation vat or vessel is used for the reactor. Alternatively, other systems can be set up if a byproduct or product such as pyruvate or ethanol is monitored instead of or in addition to the CO₂. The fermentation process is modeled using the equations as disclosed herein.

Fermentation is a metabolic process that converts sugar to acids, gases and/or alcohol. The process to produce an alcoholic beverage, such as beer, is generally irreversible and open for the CO₂ production, but closed for the production of ethanol and pyruvate. A very simple fermentation process is the conversion of glucose to ethanol and CO₂, but other processes can also occur depending on the starting carbohydrates and the process conditions. This conversion of glucose is given by the following chemical equation, where each molecule of glucose produces two molecules of ethanol and two molecules of carbon dioxide:

C₆H₁₂O₆→2C₂H₅OH+2CO₂

Because yeast are used in the fermentation process, the yeast growth is modeled via the following equations, 11 and 12.

Y=a(t)Ae^{t/T}^{ₐ}+b(t)Be^{t/T}^{_{b}}+c(t)Ce^{t/T}^{_{c}}+d(t)De^{t/T}^{_{d}} (equation 11)

Y=f(lag+exponential growth+stationary phase+death) (equation 12)

Where activity is defined for the lag in growth “(a)”, exponential growth “(b)”, stationary phase “(c)”, and death “(d)”, of the microbial organisms involved in the fermentation, to form a linear combination, where each of the terms (a), (b), (c), and (d) is a function of time: a(t), b(t), c(t), and d(t), and where A, B, C, and D are constant amplitude terms. The term “t” is the time since the inception of the fermentation reaction where Tₐ, T_{b}, T_{c}, and T_{d} define the characteristic 1/e folding, that is the decrease as a function of time.

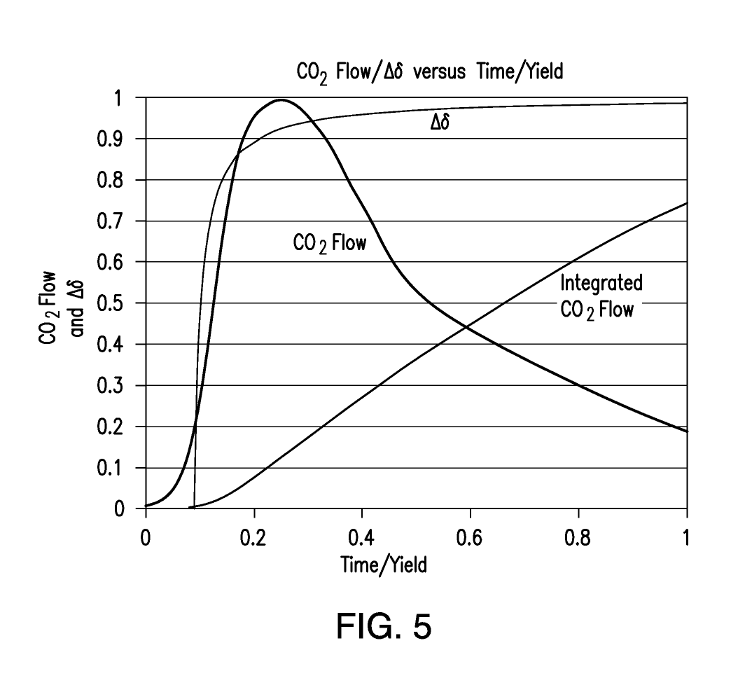

The reaction for the beer fermentation is irreversible. The reaction is open for CO₂, but closed for pyruvate (namely the conjugate base of pyruvic acid) and ethanol. The relative isotopic abundance of ¹³C is monitored as the fermentation progresses. FIG. 5 depicts graphically model equations for CO₂ flow, Δδ, and integrated yield for a beer fermentation process in which CO₂ is a by-product of the fermentation process. It is noted that (i) the Δδ vs f equation is one continuous equation (Δδ=(−1/75(f−0.075))+1), (ii) the CO₂ flow is continuous for t<0.25 (y=(1+exp(−((t*40−5))), while for t>0.25 a similar sigmoidal curve is adjusted by set of set of constants to better fit the observed relationship, and (iii) the integrated CO₂ flow graph is an integration-by-parts of the preceding CO₂ flow curve.

In this example, the progress of the fermentation reaction or process can be continuously monitored from the gaseous CO₂ produced (generally considered a byproduct of the fermentation), the pyruvate produced (generally considered a soluble byproduct of the fermentation), and the ethanol produced (generally considered a desirable product of the fermentation, because beer is an alcoholic beverage intended for consumption). The isotopic information for one or more isotope ratios from elements present in the CO₂, the ethanol, and/or the pyruvate can be determined from the ¹³C/¹²C and/or ¹⁸O/¹⁶O isotope ratios in the CO₂, and from the ²H/¹H, ¹³C/¹²C, and/or ¹⁸O/¹⁶O ratios in the pyruvate and/or ethanol. For practical reasons, including for example the relatively high natural abundance of ¹³C and ¹²C, and the relatively large dynamic range associated with their measurements, determination of the ¹³C/¹²C ratio is generally most convenient. Alternatively, other products and/or byproducts of the fermentation process and other isotope ratios can be used for monitoring the fermentation process.

The method and system described herein are useful for continuously monitoring a beer fermentation process and is generally applicable to other fermentation processes.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

1. A method for continuously monitoring the progress of a chemical process or a biological process comprising the steps of:
(a) sampling the process at a selected frequency to obtain one or more samples from the process,
(b) determining the isotopic information for one or more isotope ratios from elements present in each sample,
(c) processing the information to determine the isotopic information as a function of time, and
(d) determining the progress of the process from the information processed in step (c).

2. A method for continuously monitoring the progress of a chemical or biological process comprising the steps of:
(a) initially sampling the process at a first time point,
(b) determining the isotopic information for one or more isotope ratios from elements present in the sample obtained at the first time point,
(c) further sampling the process at one or more time points after the first time point,
(d) determining the isotopic information for one or more isotope ratios from elements present in each sample obtained after the first time point, and
(e) comparing the isotopic information from each sample obtained after the first time point with the isotopic information from the sample obtained at the first time point to determine the progress of the process.

3. A method for continuously assessing the yield of a chemical process or a biological process comprising the steps of:
(a) sampling the process at a selected frequency to obtain one or more samples from the process,
(b) determining the isotopic information for one or more isotope ratios from elements present in each sample,
(c) processing the information to determine the isotopic information as a function of time, and
(d) determining the yield of the process from the information processed in step (c).

4. A method for continuously assessing the yield of a chemical process or a biological process comprising the steps of:
(a) initially sampling the process at a first time point,
(b) determining the isotopic information for one or more isotope ratios from elements present in the sample obtained at the first time point,
(c) further sampling the process at one or more time points after the first time point,
(d) determining the isotopic information for one or more isotope ratios from elements present in each sample obtained after the first time point, and
(e) comparing the isotopic information from each sample obtained after the first time point with the isotopic information from the sample obtained at the first time point to assess the yield of the process.

5. A method according to claim 3 wherein the yield is an incremental yield.

6. A method according to claim 3 wherein the yield is an instantaneous yield.

7. A method for continuously monitoring the fraction of a reactant remaining in a chemical process or a biological process comprising the steps of:
(a) sampling the process at a selected frequency to obtain one or more samples from the process,
(b) determining the isotopic information for one or more isotope ratios from elements present in each sample,
(c) processing the information to determine the isotopic information as a function of time, and
(d) determining the amount of the reactant remaining from the information processed in step (c).

8. A method for continuously monitoring the fraction of a reactant remaining in a chemical process or a biological process comprising the steps of:
(a) initially sampling the process at a first time point,
(b) determining the isotopic information for one or more isotope ratios from elements present in the sample obtained at the first time point,
(c) further sampling the process at one or more time points after the first time point,
(d) determining the isotopic information for one or more isotope ratios from elements present in each sample obtained after the first time point, and
(e) comparing the isotopic information from each sample obtained after the first time point with the isotopic information from the sample obtained at the first time point to determine the amount of the reactant remaining.

9.-20. (canceled)

21. A method according to claim 1 wherein the elements are selected from elements that have two or more isotopes.

22. A method according to claim 1 wherein the elements are selected from hydrogen, carbon, nitrogen, oxygen, sulfur, chlorine, bromine, and combinations thereof.

23. A method according to claim 21 wherein the isotopes are stable isotopes.

24. A method according to claim 23 where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

25. A method according to claim 24 wherein the isotope ratios are selected from the following pairs of isotopes: ¹H and ²H, ¹²C and ¹³C, ¹⁴N and ¹⁵N, ¹⁶O and, ¹⁸O, ³²S and ³⁴S, ³⁵Cl and ³⁷Cl, and ⁷⁹Br, and ⁸¹Br.

26. A method according to claim 24 wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

27.-33. (canceled)

34. A method according to claim 1 wherein the isotopic information is intrinsic isotopic information.

35. A system for continuously monitoring the progress of a chemical process or a biological process comprising:
(a) a device for sampling the process,
(b) an interface,
(c) an isotope analyzer, and
(d) a computerized data system (CDS).

36. A system for continuously monitoring the progress of a chemical process or a biological process comprising:
(a) a reactor,
(b) a device for sampling the process,
(c) an interface,
(d) an isotope analyzer, and
(e) a computerized data system (CDS).

37. A system for assessing the yield of a chemical process or a biological process comprising:
(a) a device for sampling the process,
(b) an interface,
(c) an isotope analyzer, and
(d) a computerized data system (CDS).

38. A system for assessing the yield of a chemical process or a biological process comprising:
(a) a reactor,
(b) a device for sampling the process,
(c) an interface,
(d) an isotope analyzer, and
(e) a computerized data system (CDS).

39. A system according to claim 38 wherein the yield is an incremental yield.

40. A system according to 38 wherein the yield if an instantaneous yield.

41. A system for continuously monitoring the fraction of a reactant remaining in a chemical process or a biological process comprising:
(a) a device for sampling the process,
(b) an interface,
(c) an isotope analyzer, and
(d) a computerized data system (CDS).

42. A system for continuously monitoring the fraction of a reactant remaining in a chemical process or a biological process comprising:
(a) a reactor,
(b) a device for sampling the process,
(c) an interface,
(d) an isotope analyzer, and
(e) a computerized data system (CDS).

43. A system according to claim 35 wherein the device for sampling the process samples the process at a selected frequency to obtain one or more samples from the process.

44. A system according to claim 35 wherein the isotope analyzer is used to determine the isotopic ratio information for one or more isotope ratios from elements present in each sample.

45. A system according to claim 35 wherein the isotope analyzer is selected from a cavity ring-down spectrometer (CRDS), an isotope-ratio mass spectrometer (IRMS), or a nuclear magnetic resonance (NMR) spectrometer.

46.-48. (canceled)

49. A system according to claim 35 wherein the computerized data system (CDS) is used for analyzing the output from the isotope analyzer.

50. A system according to claim 49, wherein the computerized data system further stores and displays the output analyzed from the isotope analyzer.

51. The system according to claim 44 further comprising a feedback loop operably connected to the computer data system to adjust process parameters in the process using defined routines if the isotopic information is outside acceptable ranges.

52.-63. (canceled)

64. A system according to claim 44 wherein the elements are selected from elements that have two or more isotopes.

65. A system according to claim 64 wherein the elements are selected from hydrogen, carbon, nitrogen, oxygen, sulfur, chlorine, bromine, and combinations thereof.

66. A system according to claim 65 wherein the isotopes are stable isotopes.

67. A system according to claim 66 where the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

68. A system according to claim 67 wherein the isotope ratios are selected from the following pairs of isotopes: ¹H and ²H, ¹²C and ¹³C, ¹⁴N and ¹⁵N, ¹⁶O and, ¹⁸O, ³²S and ³⁴S, ³⁵Cl and ³⁷Cl, and ⁷⁹Br, and ⁸¹Br.

69. A system according to claim 67 wherein the isotope ratios are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

70.-76. (canceled)

77. A system according to claim 44 wherein the isotopic information is intrinsic isotopic information.

78. (canceled)

79. A method according to claim 1 wherein the process produces CO₂ or CO and wherein the isotopic information is obtained from CO₂ or CO produced from the process.

80.-92. (canceled)

93. A method for continuously monitoring the progress of a chemical process or a biological process which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

94. A method for continuously assessing the yield of a chemical process or a biological process which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

95. A method for continuously monitoring the progress to the end point of a chemical process or a biological process which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

96. A method for continuously monitoring the fraction of a reactant remaining in a chemical process or a biological process, which consumes a gaseous reactant or produces a gaseous product or byproduct, wherein the gaseous reactant, product or byproduct is selected from CO₂, CO and mixtures thereof, comprising the steps of:
(a) continuously sampling the gaseous reactant or the gaseous product or byproduct from the process,
(b) continuously determining or assessing the isotopic information for one or more isotope ratios from elements present in each sample or from the continuous sampling, wherein the isotope ratios are selected from ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

97. A method for continuously monitoring the proportions of two or more products produced in a chemical process or a biological process comprising the steps of:
(a) continuously sampling the products from the process,
(b) continuously determining or assessing the isotopic information for one or more isotope ratios from elements present in each sample from the products or from the continuous sampling of the products,
(c) continuously processing the information to determine the isotopic information as a function of time, and
(d) continuously determining or assessing the proportions of two or more products from the information processed in step (c).

98.-101. (canceled)

102. A method for continuously monitoring the progress of a fermentation process which produces one or more products or byproducts selected from CO₂, pyruvic acid, ethanol, and mixtures thereof comprising the steps of:
(a) continuously sampling one or more of the products or byproducts from the process,
(b) continuously determining or assessing the isotopic information for one or more isotope ratios from elements present in each sample or from the continuous sampling, wherein the isotope ratios are selected from ²H/¹H, ¹³C/¹²C, ¹⁸O/¹⁶O, and combinations thereof,
(c) continuously processing the information to determine the isotopic information as a function of time, and
(d) continuously determining or assessing the progress of the process from the information processed in step (c).

103. A method according to claim 102 wherein the byproduct is CO₂ and the isotope ratio is selected from ¹³C/¹²C, ¹⁸O/¹⁶O, and combinations thereof.

104.-108. (canceled)

109. A method according to claim 102 wherein the fermentation process is a beer fermentation process.
